(19) United States
(12) Patent Application Publication
(10) Pub. No.: US 2008/0045889 A1
(43) Pub. Date: Feb. 21, 2008

(54) Botoxo Needle Injector

(76) Inventor: Scott J. Gerondale, Newbury Park, CA (US)

Correspondence Address: ALLERGAN, INC., 2525 DUPONT DRIVE, T2-7H, IRVINE, CA, 92612-1599, US

(21) Appl. No.: 11/571,552

(22) PCT Filed: Jan. 20, 2005
(86) PCT No.: PCT/US05/01940

§ 371 Date: Apr. 30, 2007

Related U.S. Application Data
(60) Provisional application No. 60/589,946, filed on Jul. 21, 2004.
(60) Provisional application No. 60/589,655, filed on Jul. 21, 2004.
(60) Provisional application No. 60/591,506, filed on Jul. 27, 2004.
(60) Provisional application No. 60/539,922, filed on Jan. 28, 2004.

(51) Int. Cl. A61M 5/178

(52) U.S. Cl. 604/91

(57) ABSTRACT

A needle injector includes a syringe body along with a needle disposed in fluid communication with said syringe body. A plunger including a plunger head is disposed within said syringe body and a saline solution is disposed in the syringe body. A dried medicament is disposed on an inside surface of the syringe body in a position enabling movement of the plunger head in the syringe body to cause reconstitution of the medicament saline solution and ejection of the reconstituted medicament through the needle.

### CROSS REFERENCE TO RELATED APPLICATIONS

This is a national stage application under 35 U.S.C. § 371 of PCT application PCT/US 2005/019410, filed May 31, 2005, which claims the benefit of Provisional application No. 60/589,946, filed on Jul. 21, 2004; Provisional application No. 60/589,655, filed on Jul. 21, 2004; and Provisional application No. 60/591,506, filed on Jul. 27, 2004.

The present invention generally relates to a disposable syringe of the prefilled type. More particularly, the present invention relates to a prefilled, disposable syringe assembly wherein medicament, such as BOTOX®, and diluent separately contained in the syringe. When it is desired to mix the components, a plunger is moved in order to cause intermixing of the components.

### SUMMARY OF THE INVENTION

Generally, a needle injection in accordance with the present invention includes a syringe body along with a needle disposed in fluid communication with the syringe body and a plunger, with a plunger head disposed within said syringe body.

A saline solution is disposed in said syringe body along with a dried medicament, such as BOTOX®, which is disposed on an inside surface of said syringe body in a position enabling movement of the plunger head in said syringe body to cause reconstitution of the medicament in the saline solution and ejection of the reconstituted medicament through the needle.

In one embodiment of the present invention, needle injector generally includes a syringe body and a needle disposed for fluid communication with the syringe body along with a rupturable membrane disposed between the needle and the syringe body and a plunger, including a plunger head. The plunger head, operable by the plunger, is slidably and sealably engaged with an inside surface of the syringe body and initially disposed within the syringe body at position providing a forechamber and an aftchamber within the syringe body.

A saline solution is disposed in the forechamber and the plunger head prevents transfer of the same saline solution from the forechamber into the aftchamber.

A dried medicament, preferably BOTOX®, is disposed on the inside surface of the syringe body within the aftchamber. The dried medicament has a thickness enabling the plunger head to pass thereover during withdrawal of the plunger from the syringe body, thus causing reconstitution of the medicament in the saline solution.

Thereafter, insertion of the plunger into the syringe body causes the reconstituted medicament to be injected through the needle.

More particularly, the syringe body may be provided with an abrasive perimeter for engaging the inside surface of the syringe body to agitate the dried BOTOX® in order to promote introduction into the saline solution and accordingly reconstitution of the BOTOX® in the saline solution.

In another embodiment of the present invention the needle injector generally includes a syringe body having a first end and a second end with a cylinder extending between the first and second ends. The cylinder includes a bypass disposed between the first and second ends.

A needle is provided and coupled to the syringe body first end and is in fluid communication with the syringe body. A dried medicament is disposed in the bypass and a slidable stopper is disposed between the bypass and the syringe body second end.

A plunger is provided including a plunger head with the plunger head slidably and sealably engaging in an inside surface of the syringe body cylinder and initially disposed within the syringe body proximate the syringe body second end.

A saline solution is disposed within the syringe body between the slidable stopper and the plunger head.

The plunger, and plunger head, are movable within the syringe body from proximate the syringe body second end toward the syringe body first end. This movement forces the stopper toward the syringe body front end and the bypass enables flow of the saline solution pass the stopper when the stopper is aligned with the bypass. The flow of saline solution through the bypass causes reconstitution of the dried medicament in the saline solution.

Further forward movement of the plunger head causes engagement with the stopper after all of the saline solution flows through the bypass. Continued movement of the plunger head and stopper causes ejection of the reconstituted medicament through the needle. The dried medicament again is preferably botulinum toxin.

In yet another embodiment of the present invention, the needle injector generally includes a syringe body having a first end and a second end with a cylinder extending between the first and second ends. A slidable piston including a permeable septum is disposed in the cylinder between the first and second ends.

A needle is provided and coupled to the syringe body first end and is in fluid communication with the syringe body, dried medicament is disposed in the cylinder between the syringe body first end and the piston, and a slidable stopper is disposed in the cylinder been the first and second ends.

A plunger is provided including a plunger head with the plunger head slidably and sealably engaging in an inside surface of the syringe body cylinder and initially disposed within the syringe body proximate the syringe body second end.

A saline solution is disposed within the syringe body between the slidable piston and the plunger head. The stopper may be sized in order to provide sealing fractional engagement with the syringe body inside surface or, alternatively, may include wings for releasably engaging an enlarged diameter of the cylinder to prevent premature movement of the stopper and provide sufficient resistance to movement for the enabling saline solution to pass therethrough as hereinafter discussed in greater detail.

The plunger, and plunger head, are movable within the syringe body from proximate the syringe body second end toward the syringe body first end. This movement forces the saline solution through the permeable septum toward the syringe body first end. The flow of saline solution through the septum enables reconstitution of the dried medicament in the saline solution.

Further forward movement of the plunger head causes engagement with the piston after all of the saline solution flows therethrough. Continued movement of the plunger head and piston causes ejection of the reconstituted medicament through the needle. The dried medicament is preferably botulinum toxin.

### BRIEF DESCRIPTION OF THE DRAWINGS

The present invention may be more clearly understood with reference to the appended drawings of which:

FIG. 1 is a perspective view of one embodiment of a needle injector in accordance with the present invention partially broken away and showing generally a syringe body, plunger along with a plunger head, saline solution and a dried medicament;

FIG. 2 is a view similar to FIG. 1 showing withdrawal of the plunger and reconstitution of medicament in the saline solution;

FIG. 3 is a view similar to FIGS. 1 and 2 showing insertion of the plunger and dispensement of reconstituted medicament through the needle;

FIG. 4 is a perspective view of another embodiment of a needle injector in accordance with the present invention partially broken away showing a syringe body, a needle, a stopper, a plunger, plunger head, dried medicament, and a saline solution;

FIG. 5 is a cross sectional partial view of the injector shown in FIG. 4 showing operation of the injector for reconstituting the dried medicament in the saline solution, as illustrated, saline solution flows from between the stopper and the plunger head through the bypass when the stopper is aligned with the bypass;

FIG. 6 is a view similar to that shown in FIG. 5 showing all of the saline solution transferred to in front of the stopper with continued movement of the stopper and engaging plunger head causing ejection of the saline solution through the needle, not shown in FIG. 6;

FIG. 7 is a perspective view of yet another embodiment of a needle injector in accordance with the present invention partially broken away showing a syringe body, a needle, a piston, a plunger, plunger head, dried medicament, and a saline solution;

FIG. 8 is a cross sectional partial view of the injector shown in FIG. 7 showing operation of the injector for reconstituting the dried medicament in the saline solution, as illustrated, saline solution flows from between the piston and the plunger head through the piston; and

FIG. 9 is a view similar to that shown in FIG. 8 showing all of the saline solution transferred to in front of the piston with continued movement of the plunger head engaging piston causing ejection of the saline solution through the needle, not shown in FIG. 9.

### DETAILED DESCRIPTION

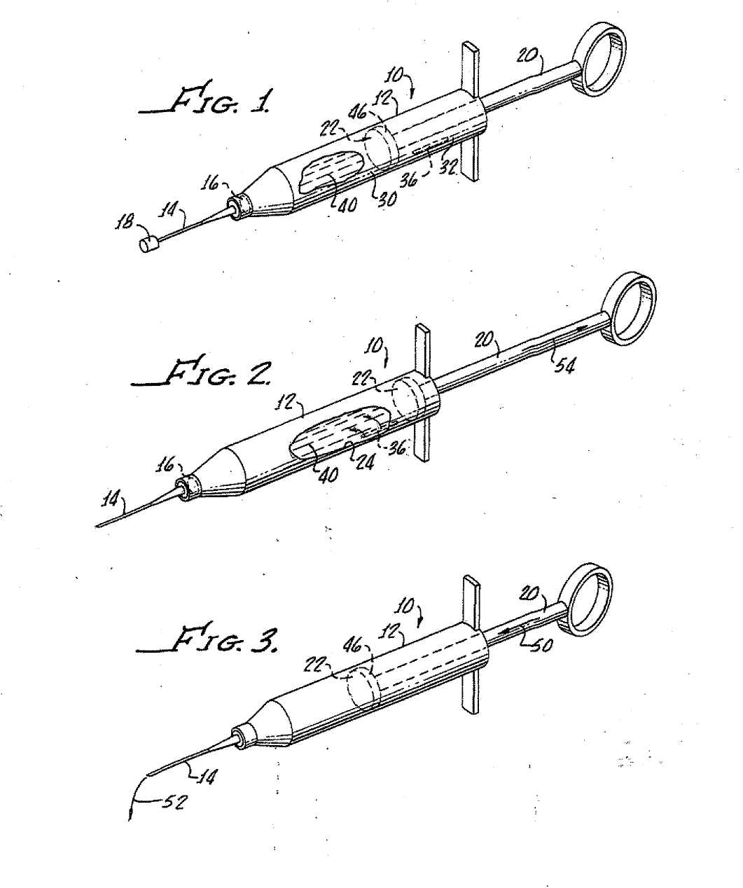

With reference to FIG. 1, there is shown a needle injector 10 in accordance with the present invention generally including a syringe body 12 and a needle 14 disposed for fluid communication with a syringe body. A rupturable membrane 16 may be provided or a cap 18 utilized to prevent premature loss of saline solution through a needle 14.

A plunger 20 includes a plunger head 22 slidably and sealably engaging an inside surface 24 of the syringe body 12.

As shown in FIG. 1, the plunger head 24 is initially disposed within the syringe body 12 for defining and providing a forechamber 30 and an aftchamber 32.

A dried medicament 36, preferably BOTOX®, in an amount of perhaps 100 microliters is deposited on the inside surface 24.

The dried medicament has a thickness of, for example, about a few mils or less. With the use of a syringe body 12 having a diameter of between about 118 mils and about ½ inch, the plunger head 22 passes over the dried medicament during withdrawal of the plunger 20 from the syringe body 12 as shown in FIG. 2 thus reconstituting the BOTOX® medicament in a saline solution 40 initially stored in the forechamber 30.

The plunger head 22 initially prevents transfer of the saline solution 40 from the forechamber 30 into the aftchamber 32.

Withdrawal of the plunger 20 and plunger head 22 exposes the medicament 36 to the saline solution 40, thus enabling reconstitution of the medicament 36 into the saline solution 40. It should be appreciated that a perimeter 46 of the plunger head 22 may include an o-ring or a otherwise roughened surface in order to agitate the medicament 36 upon a passage thereover as is shown in FIG. 2 to enhance, or promote, physical introduction of the medicament 36 into the solution to distribute the medicament in the saline solution for dissolution thereinto, thus effecting reconstitution of the medicament 36.

After reconstitution, as shown in FIG. 3, the plunger 20 is force forward in the syringe body 12, as indicated by the arrow 50, thus causing the reconstituted medicament to be ejected through the needle 14 after rupture of the membrane 16, as indicated by the arrow 52.

It should be appreciated that the membrane 16 may be configured for rupture, as the plunger 20 is withdrawn from the syringe body 12, as shown in FIG. 2 by arrow 54. However, rupture of the membrane 16 is insured during insertion of the plunger 20 and plunger head 22 into the syringe body as shown in FIG. 3 in order to cause ejection of reconstituted medicament, as illustrated by the arrow 52.

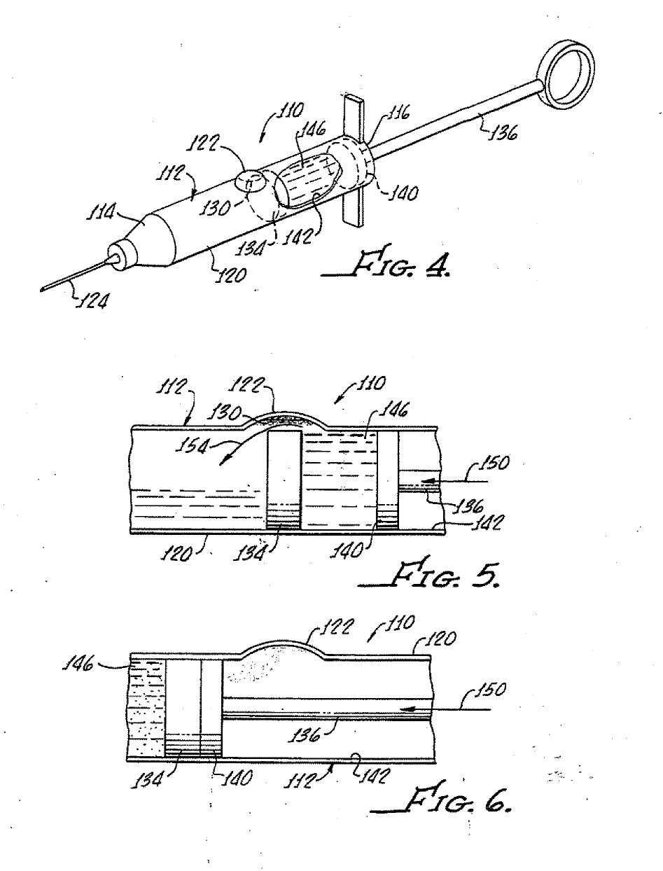

With reference to FIGS. 4, 5, and 6, there is shown a needle injector 110 in accordance with the present invention generally including a syringe body 112 having a first end 114 and a second end 116 along with a cylinder 120 extending therebetween. A bypass 122, more clearly shown in FIGS. 5 and 6, is provided in the cylinder 112 between the first end 114 and second end 116.

A needle 124 is coupled to the syringe body front end 114 and is in fluid communication with the syringe body 112. A dried medicament 130, such as botulinum toxin, is disposed in the bypass 122.

A slidable stopper 134 is disposed between the bypass 122 and the syringe body second end 116 and a plunger 136 including a plunger head 140 is provided with the plunger head 140 slidably and sealably engaging an inside surface 142 of the syringe body 112. The plunger head 140 is initially disposed proximate the syringe body second end 116, as shown in FIG. 4, with a saline solution 146 disposed between the slidable stopper 134 and plunger head 140.

In operation, as shown in FIGS. 5 and 6, the plunger 136 is moved toward the syringe body first end 114, as shown by a arrow 150. Due to the incompressibility of the saline solution 146, the slidable stopper 134 also moves forward toward the syringe body first end 114. However, when the slidable stopper 134 reaches the bypass, as shown in FIG. 5, frictional engagement between the slidable stopper 134 and the surface 142 enables saline solution to flow through the bypass 122 as indicated by the arrow 154. The saline solution passing over the dried medicament 130 causes a reconstitution of the medicament 130 in the saline solution which then is forced between the stopper 134 and the syringe body first end 114 by continued movement of the plunger head 140 toward the syringe body first end 114 and stopper 134, as shown in FIG. 6. Disposing the medicament in the bypass enhances erosion and reconstitution in the saline solution forced therepast due to venturi effects.

Continued movement of the plunger head 140 causes engagement with the stopper 134 after passage of all of the saline solution through the bypass 132. The saline solution 146 with reconstituted medicament 134 is then ejected through the needle 124 by the stopper 134, plunger head 140 in a conventional manner.

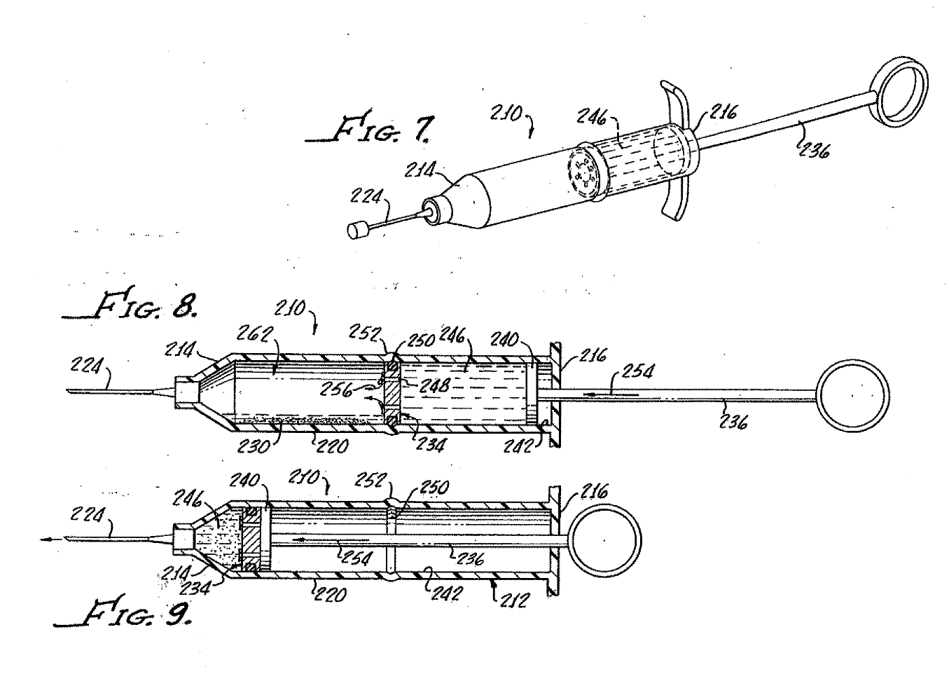

With reference now to FIGS. 7, 8, and 9, there is shown a needle injector 210 in accordance with the present invention generally including a syringe body 212 having a first end 214 and a second end 216 along with a cylinder 220 extending therebetween.

A needle 224 is coupled to the syringe body first end 214 and is in fluid communication with the syringe body 212. A dried medicament 230, such as botulinum toxin, is disposed in the cylinder 220.

A slidable piston 234 is disposed between the syringe body first and second ends 214, 216 and a plunger 236 including a plunger head 240 is provided with the plunger head 240 slidably and sealably engaging an inside surface 242 of the syringe body 212. The plunger head 240 is initially disposed proximate the syringe body second end 216, as shown in FIG. 7, with a saline solution 246 disposed between the slidable stopper 234 and plunger head 240.

As shown, the piston includes a permeable septum 248, which may be a membrane or rupturable portions of the piston 234, or valves for enabling passage of the saline solution 246 through the piston upon pressure executed by the plunger head 240. The piston may be sized and formed of a material providing sufficient engaging friction with the inner surface 242 so that the stopper remains in portion during passing of saline solution therethrough.

Alternatively, wings 250, or the like, shown in dashed line, may be provided on the stopper 234 for engaging the inner surface 242. Also, a ring 252 of enlarged cylinder 220 diameter may be provided for engaging the wings to maintain the stopper 234 in an initial position as the saline solution passes therethrough, the stopper 234 eventually being moved by engagement with the plunger head 240.

In operation, as shown in FIGS. 8 and 9, the plunger 236 is moved toward the syringe body first end 214, as shown by a arrow 254. Pressure on the saline solution 246, forces the saline solution through the piston 234 indicated by the arrow 256. The saline solution passing over the dried medicament 230 causes a reconstitution of the medicament 230 in the saline solution which then is forced between the piston 234 and the syringe body first end 214 by continued movement of the plunger head 240 toward the syringe body first end 214 and piston 234, as shown in FIG. 9.

Continued movement of the plunger head 240 causes engagement with the piston 234 after passage of all of the saline solution through the septum 248. The saline solution 246 with reconstituted medicament 234 is then ejected through the needle 224 by the piston 234, plunger head 240 in a conventional manner.

Although there has been hereinabove described specific embodiments of BOTOX® needle injectors in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A needle injector comprising:
a syringe body;
a needle disposed in fluid communication with said syringe body;
a plunger including a plunger head is disposed within said syringe body;
a saline solution disposed in said syringe body;
a dried medicament disposed on an inside surface of said syringe body in a piston enabling movement of said plunger head in said syringe body to cause reconstitution of the medicament in said saline solution and ejection of the reconstituted medicament through said needle.

2. The needle injector according to claim 1 wherein said medicament comprises botulinum toxin.

3. A needle injector comprising:
a syringe body having a first end, a second end, and a cylinder extending between the first and second ends, said cylinder having a bypass disposed between the first and second ends;
a needle coupled to the syringe body first end and in fluid communication with said syringe body;
a dried medicament disposed in said bypass;
a slidable stopper disposed between said bypass and the syringe body second end;
a plunger, including a plunger head, said plunger head slidably and sealably engaging an inside surface of said syringe body and initially disposed within said syringe body proximate a syringe body second end;
a saline solution disposed in said syringe body between said slidable stopper and said plunger head;
said plunger being movably within said syringe body from proximate the syringe body second end toward the syringe body first end, such movement forcing stopper toward the syringe body first end, said bypass enabling flow of the saline solution past the stopper and causing reconstitution of said dried medicament in said saline solution as the saline solution flow past the stopper, said plunger head engaging the stopper after all of the saline solution flow through the bypass for ejecting the reconstituted medicament through said needle.

4. The needle injection according to claim 3 wherein said medicament comprises botulinum toxin.

5. A needle injector comprising:
a syringe body having a first end, a second end, and a cylinder extending between the first and second ends;
a needle coupled to the syringe body first end and in fluid communication with said syringe body;
a slidable piston, including a permeable septum, disposed in the cylinder between the first and second ends;
a dried medicament disposed in said cylinder between the syringe body first end and the piston;
a plunger, including a plunger head, said plunger head slidably and sealably engaging an inside surface of said syringe body and initially disposed within said syringe body proximate a syringe body second end;
a saline solution disposed in said syringe body between said slidable piston and said plunger head;
said plunger being movably within said syringe body from proximate the syringe body second end toward the syringe body first end, such movement forcing the saline solution through the permeable septum and causing reconstitution of said dried medicament in said saline solution as the saline solution flows past the piston, said plunger head engaging the piston after all of the saline solution flows through the septum for ejecting the reconstituted medicament through said needle.

6. The needle injection according to claim 5 wherein said medicament comprises botulinum toxin.
